(19) United States
(12) Patent Application Publication
(10) Pub. No.: US 2021/0219882 A1
(43) Pub. Date: Jul. 22, 2021

(54) METHODS AND SYSTEMS FOR NON-INVASIVE MEASUREMENT AND MONITORING OF PHYSIOLOGICAL PARAMETERS

(71) Applicant: General Electric Company, Schenectady, NY (US)

(72) Inventors: Matti Veli Tapani Huiku, Espoo (FI); Pellervo Ruponen, Helsinki (FI); Sakari Matias Lamminmaki, Espoo (FI)

(21) Appl. No.: 17/221,720

(22) Filed: Apr. 2, 2021

Related U.S. Application Data
(63) Continuation-in-part of application No. 15/410,684, filed on Jan. 19, 2017.

(51) Int. Cl. A61B 5/1455; A61B 5/08; A61B 5/00; G16H 50/30; G16H 40/67

(52) U.S. Cl. CPC A61B 5/14552; A61B 5/0816; A61B 5/7278; A61B 5/7405; A61B 5/6819; G16H 50/30; G16H 40/67; A61B 5/7425

(57) ABSTRACT

Methods and systems are provided for a pulse oximetry probe configured to measure blood originating from the internal carotid artery. One example system includes a light emitter and a light detector coupled to a substrate and an attachment mechanism configured to couple the nasal pulse oximetry probe to a nose of a patient, the light emitter and light detector positioned on opposite sides of the nose at the root of the nasal bridge. One example method determines peripheral arterial oxygen saturation (SpO₂) respiratory variability as well as other physiological parameters from measurements taken from the internal carotid artery.

### CROSS-REFERENCE TO RELATED APPLICATIONS

The present application is a continuation-in-part of U.S. Non-Provisional patent application Ser. No. 15/410,684, entitled “PULSE OXIMETRY SENSORS AND METHODS”, and filed on Jan. 19, 2017. The entire contents of the above-listed application are hereby incorporated by reference for all purposes.

### FIELD

Embodiments of the subject matter disclosed herein relate to methods and devices for noninvasive measurement and monitoring of physiological parameters.

### BACKGROUND

Photoplethysmography (PPG) relates to the use of optical signals transmitted through or reflected by blood-perfused tissues for monitoring a physiological parameter of a subject (also referred to as a patient herein). In this technique, one or more emitters are used to direct light at a tissue, and one or more detectors are used to detect the light that is transmitted through or reflected by the tissue.

In clinical practice, the level of arterial oxygenation can be measured either directly by blood gas sampling to measure partial pressure (PaO₂) and percentage saturation (SpO₂) or indirectly through pulse oximetry. Pulse oximetry is, at present, the standard of care for continuously monitoring arterial oxygen saturation (SpO₂), one component of oxygenation. Oxygen saturation is proportional to the partial pressure of oxygen (PaO₂—also known as arterial oxygen tension). Thus, a patient's arterial oxygen saturation provides information about the amount of oxygen that is available to the metabolizing tissues through its correlation with PaO₂.

PaO₂ reflects how well oxygen is able to move from the lungs into the blood. SpO₂ is directionally, but not linearly, associated with PaO₂ and their relationship is shown in an oxygen-hemoglobin dissociation curve which plots the proportion of hemoglobin in its saturated form against the prevailing oxygen tension. The sigmoid shape of the oxygen-hemoglobin dissociation curve means that at higher ranges of partial pressure and saturation, which are in the flat portion of the curve, there is not a significant decrease in oxygen saturation as the oxygen pressure begins to fall. However, in the steep part of the curve, small fluctuations of oxygen partial pressure can lead to marked fluctuations in the measured oxygen saturation.

Conventional pulse oximetry probes are typically mounted to an extremity of the subject (e.g., a finger or ear lobe). Generally, conventional pulse oximeters are more accurate at high blood oxygen saturation with decreasing accuracy once oxygen saturation levels fall below 90%; for example, if a patient is suffering from hypoxemia or dyshemoglobinemias or other problems. Additionally, vasoconstriction, such as during times of severe physiological stress, may result in insufficient pulse amplitude for a conventional pulse oximeter to reliably measure blood or blood circulation characteristics.

### BRIEF DESCRIPTION

In one embodiment, a system for an optical probe comprises a light emitter and a light detector coupled to a substrate, the light emitter and light detector configured to measure blood originating at least partly from an internal carotid artery of a patient; and an attachment mechanism configured to couple the optical probe to a location on the head or trunk of the patient. In some aspects, the attachment mechanism is configured to couple the optical probe to a nose of the patient, the light emitter and light detector positioned to be on opposite sides of the nose of the patient at a root of a nasal bridge when the optical probe is worn by the patient.

Thus, blood from the internal carotid artery, which does not undergo substantial autonomic nervous system (ANS) regulation, may be monitored, resulting in strong PPG signals even under physiological stress conditions such as hypothermia and hypovolemia.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 is a block diagram illustrating one embodiment of a multi-wavelength pulse oximetry system.

FIG. 2A shows a cutaway side view of an example nasal pulse oximetry probe.

FIG. 2B shows a top view of the example nasal pulse oximetry probe.

FIG. 3A is a partial profile view of a human face illustrating how a nasal pulse oximetry probe may be positioned on a nose with respect to internal vasculature of the nose.

FIG. 3B is a partial profile view of a human face illustrating how a nasal pulse oximetry probe may be positioned on a nose with respect to other anatomical features of the face.

FIG. 4 is a partial front view of a face of a subject wearing a first embodiment of a nasal pulse oximetry probe.

FIG. 5 is a partial front view of a face of a subject wearing a second embodiment of a nasal pulse oximetry probe.

FIG. 6 is a high-level method for calculating physiological parameters from a PPG signal measured by a nasal pulse oximetry sensor.

FIG. 7 shows an example method for calculating SpO₂ of a patient based on the PPG signal data obtained by a nasal pulse oximetry sensor.

FIG. 8 shows an example method for calculating pulse transit time (PTT) of a patient from PPG signal data obtained by a nasal pulse oximetry sensor.

FIG. 9 shows an example method for calculating respiration rate of a patient from PPG signal data obtained by a nasal pulse oximetry sensor.

FIG. 10 shows an example method for determining if hypovolemia is indicated from PPG signal data obtained by a nasal pulse oximetry sensor.

FIG. 11 shows an example method for determining blood circulation at a measurement site from PPG signal data obtained by a nasal pulse oximetry sensor.

FIG. 12 is an example graph showing how separate SpO₂ values may be determined for systole and diastole.

FIG. 13 is an example graph showing microchanges of the saturation of arterial blood within one cardiac cycle plotted against heartbeat count.

FIG. 14 is an example graph showing fast Fourier transformed PPG signal data for a normovolemic subject and a hypovolemic subject with measurements taken via the internal carotid artery.

FIG. 15 is an example graph showing the effect of different amounts of positive end-expiratory pressure on PPG signal data obtained by a nasal pulse oximetry sensor.

FIG. 16 is an example graph showing fast Fourier transformed PPG signal data for a normovolemic subject and a hypovolemic subject with measurements taken via a finger.

FIG. 17 is an example graph showing SpO₂ respiratory variability over time during the development of hypovolemia.

FIG. 18 is an example graph showing SpO₂ respiratory variability and PPG amplitude variability over time during the development of hypovolemia in comparison to ventilator flow.

### DETAILED DESCRIPTION

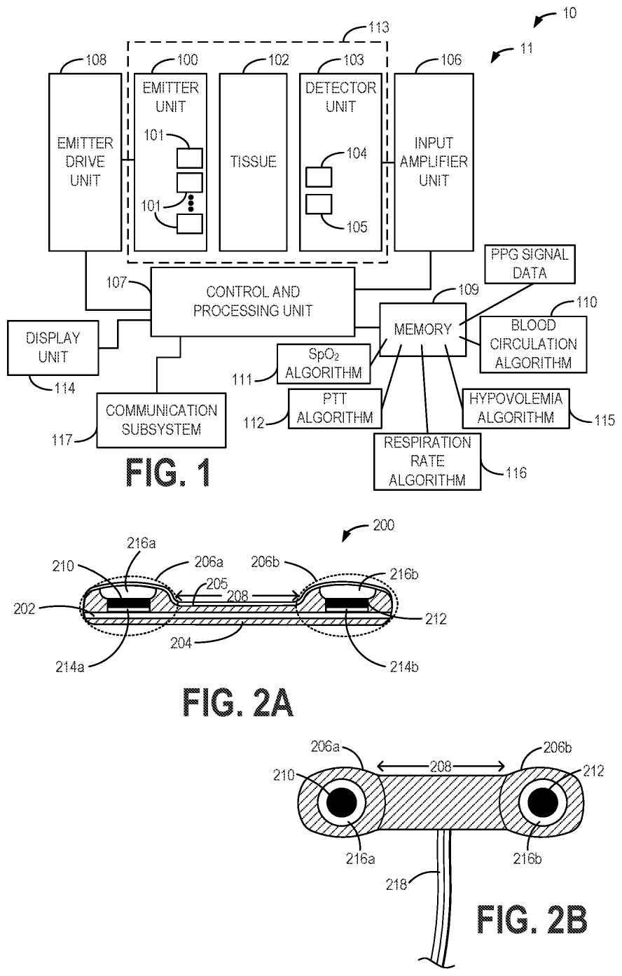

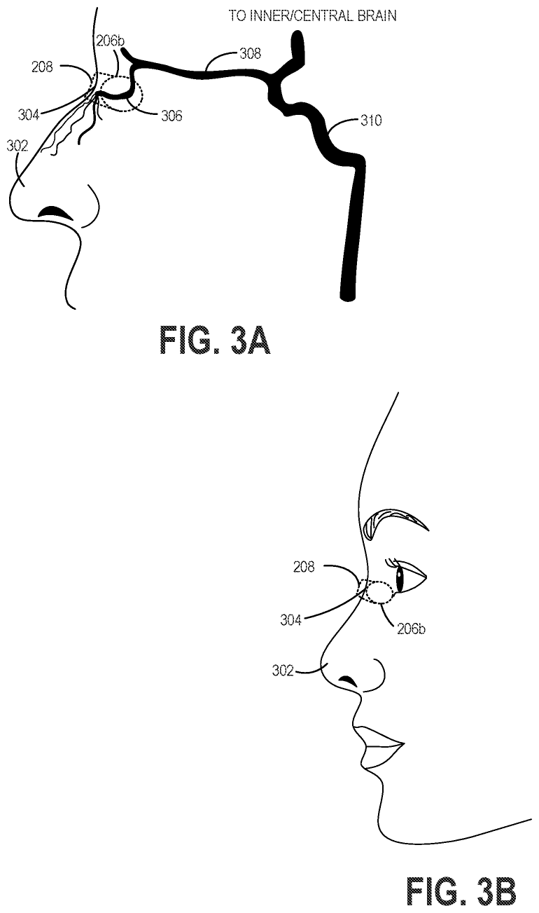

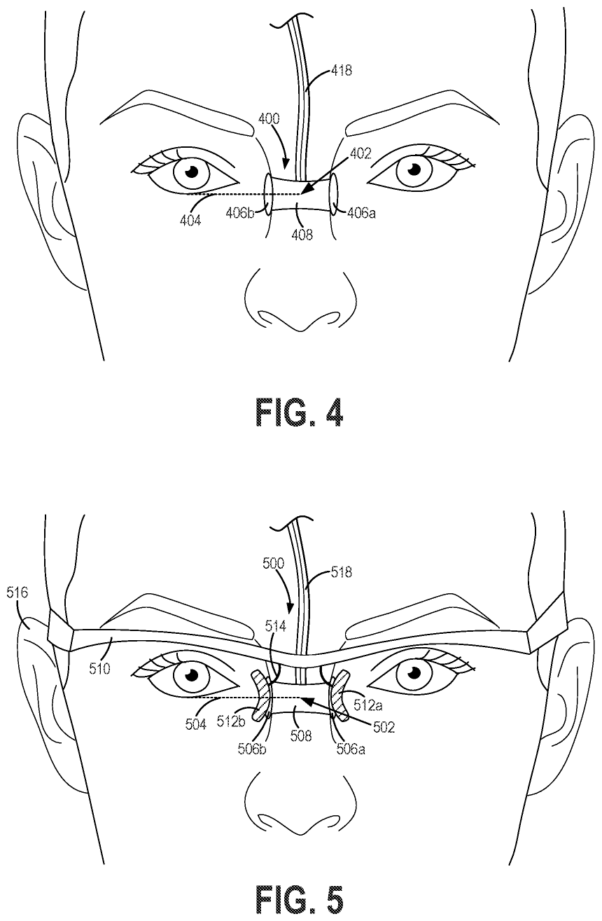

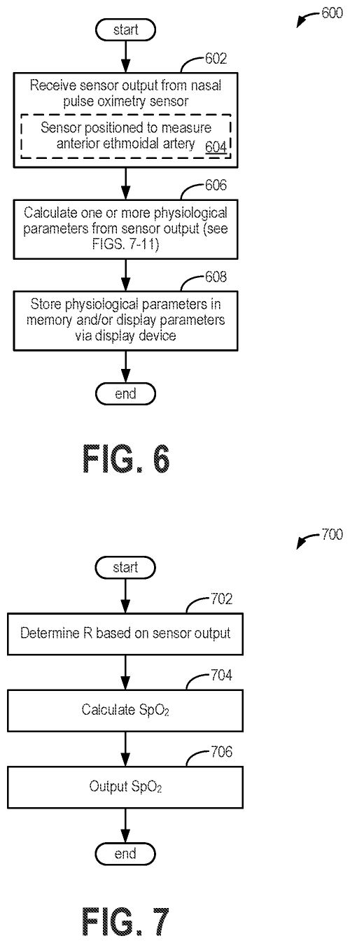

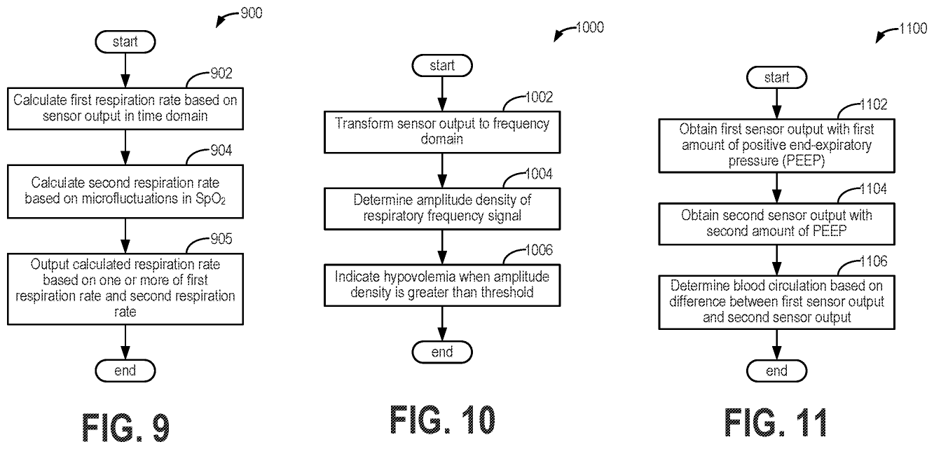

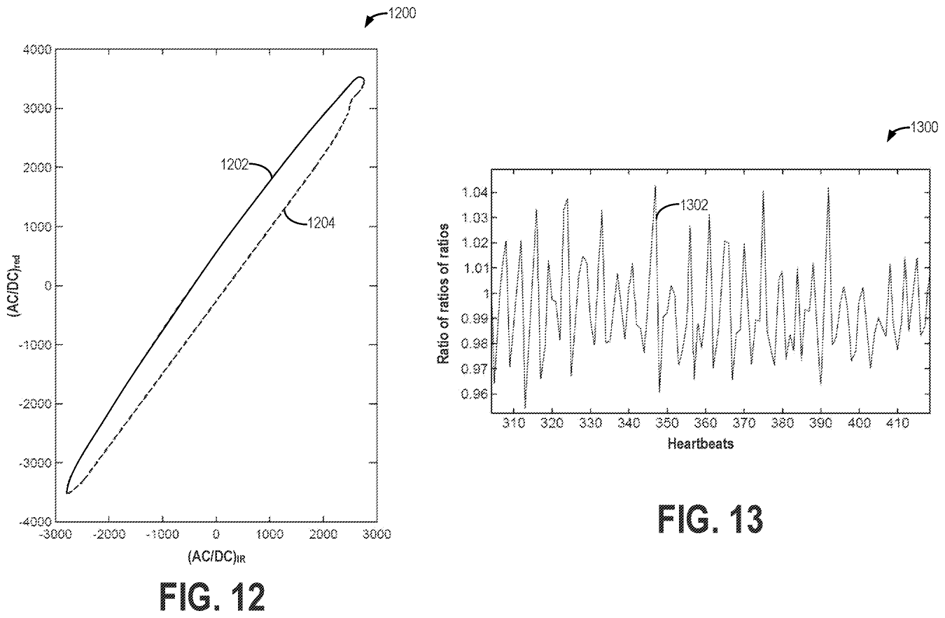

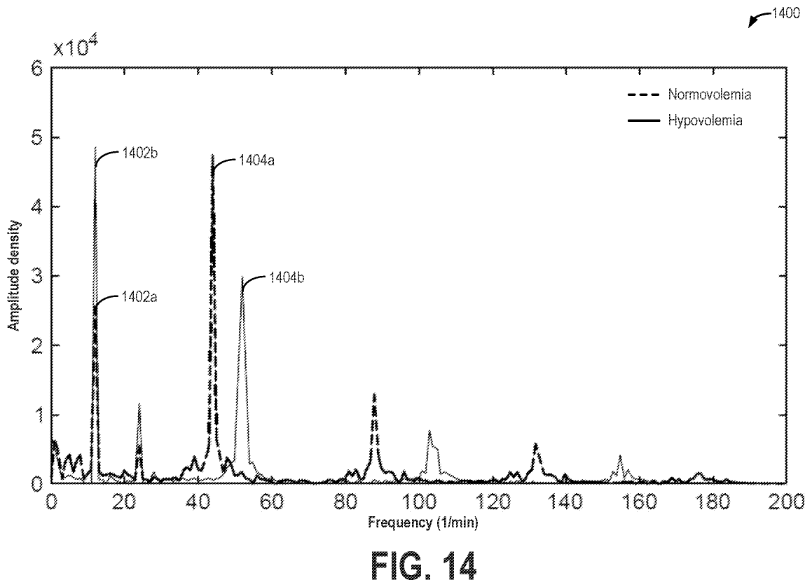

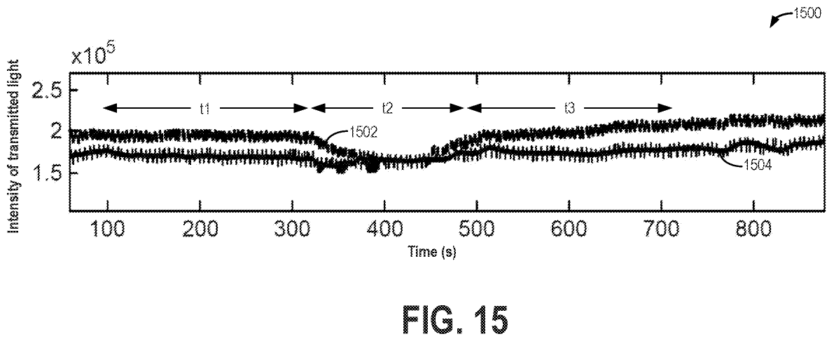

The following description relates to various embodiments of an optical probe, such as the nasal pulse oximetry probe shown in FIGS. 2A and 2B. The probe may be included in a pulse oximetry sensor or system, such as the system of FIG. 1, for determining physiological parameters of a patient. The probe is configured to be positioned on a root of a nasal bridge of the patient, as illustrated in FIGS. 3A-3B, to measure blood of an anterior ethmoidal artery. According to one embodiment of the invention, the probe may be attached to the patient without an external pressure element, as shown in FIG. 4. According to another embodiment of the invention, the probe may be attached to the patient with an added pressure element, as shown in FIG. 5. Output of the probe (e.g., a PPG waveform) may be used to calculate physiological parameters, for example, using the methods of FIGS. 6-11. In particular, respiration rate may be determined in more than one way due to strong respiratory-related variation measured in blood of the anterior ethmoidal artery, as described in the method of FIG. 9, and illustrated in the graphs of FIGS. 12-13. Further, the graphs of FIGS. 14, 16, 17, and 18 illustrate how the respiratory-related variation may be used to determine hypovolemia in the patient, as also described in the method of FIG. 10. The graph of FIG. 15 illustrates an example of the effect of positive end-expiratory pressure (PEEP) on the PPG waveform, which may be used to determine blood circulation, for example, according to the method of FIG. 11.

A pulse oximeter comprises a computerized measuring unit and a probe attached to a patient, typically a finger or ear lobe of the patient. Conventional pulse oximetry probes sequence through one LED on, then the other, then both off. The sequence is repeated typically with a frequency of several hundred Hz. The probe includes a light source for sending an optical signal through tissue of the patient and a photo detector for receiving the signal transmitted through or reflected from the tissue. On the basis of the transmitted and received signals, light absorption by the tissue may be determined.

During each cardiac cycle, light absorption by the tissue varies cyclically, that is, there is a heartbeat related change in light transmission. During the diastolic phase, absorption is caused by venous blood, non-pulsating arterial blood, cells and fluids in tissue, bone, and pigments. The level of light transmitted at the end of the diastolic phase is typically referred to as the “DC component” of the total light transmission. The DC component captures transmission through tissue, capillary blood, venous blood, and the non-pulsatile arterial blood. During the systolic phase, there is an increase in light absorption (e.g., a decrease in transmitted light) compared with the diastolic phase due to the inflow of arterial blood into the tissue on which the pulse oximetry probe is attached. A crucial pulse oximetry principle is how the measurement can be focused on the blood volume representing the arterial blood only. In pulse oximetry, this is done by taking the pulsating arterial blood component (the “AC signal”) from the total transmission signal and normalizing this signal by the “DC” component. The resulting “AC/DC” signal is called the PPG waveform. Pulse oximetry is thus based on the assumption that the pulsatile component of the absorbance is due to arterial blood only.

In pulse oximetry, arterial blood is typically modeled as containing two species of hemoglobin:oxyhemoglobin (HbO₂) and reduced hemoglobin (Hb). Oxyhemoglobin is hemoglobin that is fully saturated with oxygen, and reduced hemoglobin is without oxygen. Arterial oxygen saturation measured by pulse oximetry (SpO₂) is defined as the percentage of HbO₂ divided by the total amount of hemoglobin (HbO₂+Hb).

In order to distinguish between the two species of hemoglobin, light absorption is measured at two different wavelengths. The probe of a traditional pulse oximeter includes two different light sources, such as light-emitting diodes (LEDs) or lasers, that emit light at two different wavelengths. Each light source is illuminated in turn at a frequency that is typically several hundred Hz. The wavelength values widely used are 660 nm (red light) and 900 nm (infrared light), as the two species of hemoglobin have substantially different absorption at these wavelengths. As oxygenated hemoglobin absorbs more infrared light and allows more red light to pass through and deoxygenated hemoglobin allows more infrared light to pass through and absorbs more red light, the ratio of the red light measurement to the infrared light measurement may be used to measure SpO₂. The SpO₂ value may be used alone or in conjunction with a plethysmograph waveform, a measurement of volumetric changes associated with pulsatile arterial blood flow which is used to ensure reliability of the calculated oxygen saturation.

FIG. 1 is a block diagram of one embodiment of a multi-wavelength pulse oximetry system 10. Light transmitted from an emitter unit 100 passes into patient tissue 102. The emitter unit includes multiple light sources 101, such as light-emitting diodes (LEDs), with each light source having a dedicated wavelength. Each wavelength forms one measurement channel on which PPG waveform data are acquired. The number of sources/wavelengths is at least two.

The light transmitted through the tissue 102 is received by a detector unit 103, which comprises two photo detectors 104 and 105 in this example. For example, photo detector 104 may be a silicon photodiode, and photo detector 105 may be a second silicon photodiode with different spectral characteristics or an indium gallium arsenide (InGaAs) photodiode. The emitter and detector units form a probe detector subunit 113 of the pulse oximetry system 10. The photo detectors convert the optical signals received into electrical pulse trains and feed them to an input amplifier unit 106. The amplified measurement channel signals are further supplied to a control and processing unit 107, which executes instructions stored in memory to convert the signals into digitized format for each wavelength channel.

The control and processing unit 107 further controls an emitter drive unit 108 to alternately activate the light sources. As mentioned above, each light source is typically illuminated several hundred times per second. With each light source being illuminated at such a high rate compared to the pulse rate of the patient, the control and processing unit 107 obtains a high number of samples at each wavelength for each cardiac cycle of the patient. The value of these samples varies according to the cardiac cycle of the patient. In some aspects, each individual SpO₂ measurement in the series of SpO₂ measurements may be taken during a duration which is equal to a cardiac interbeat interval. In other aspects, the duration during which each individual SpO₂ measurement is taken is less than a cardiac interbeat interval and thus a series of SpO₂ measurements may be taken during a cardiac interbeat interval.

The input amplifier unit 106, the control and processing unit 107, the emitter drive unit 108, and probe detector subunit 113 collectively form a probe 11. As used herein, the term “probe” may refer to the probe 11 and the attachment parts that attach the optical components, the probe 11, to the tissue site. The term “SpO₂ sensor” may refer to a unit comprising a probe, an analog front end, and a signal processing unit that calculates SpO₂ and other blood characteristics. In a multi-parameter body area network system, the system typically represents a set of multiple sensors, e.g., the different physiological parameter measurements. Therefore, the whole measurement system may comprise of several sensors and their associated probes, and the sensors may communicate to a common hub in which the parameters' information is integrated.

The digitized PPG signal data at each wavelength may be stored in a memory 109 of the control and processing unit 107 before being processed further according to non-transitory instructions (e.g., algorithms) executable by the control and processing unit 107 to obtain physiological parameters. For example, memory 109 may comprise a suitable data storage medium, for example, a permanent storage medium, removable storage medium, and the like. Additionally, memory 109 may be a non-transitory storage medium. In some examples, the system 10 may include a communication subsystem 117 operatively coupled to one or more remote computing devices, such as hospital workstations, smartphones, and the like. The communication subsystem 117 may enable the output from the detector units (e.g., the digitized PPG signal data) to be sent to the one or more remote computing devices for further processing and/or the communication subsystem may enable the output from the algorithms discussed below (e.g., determined physiological parameters) to be sent to the remote computing devices. The communication subsystem 117 may include wired and/or wireless communication devices compatible with one or more different communication protocols. As non-limiting examples, the communication subsystem 117 may be configured for communication via a wireless telephone network, a local- or wide-area network, and/or the Internet.

Algorithms may utilize the same digitized signal data and/or results derived from the algorithms and stored in the memory 109, for example. For example, for the determination of oxygen saturation and pulse transit time (PTT), the control and processing unit 107 is adapted to execute a SpO₂ algorithm 111 and a PTT algorithm 112, respectively, which may also be stored in the memory 109 of the control and processing unit 107. Further, a blood circulation algorithm 110, a hypovolemia algorithm 115, and a respiration rate algorithm 116 may also be stored in the memory 109 for determining blood pressure, an indication of hypovolemia, and respiration rate, respectively. The use of such algorithms will be described in more detail below with respect to FIGS. 6-11. The obtained physiological parameters and waveforms may be shown on a screen of a display unit 114. Further, in some examples, the control and processing unit, memory, and/or other subsystems may be located remotely from the rest of the sensor on a separate device, and the signal data from the detector units may be sent to the separate device for processing.

As used herein, the terms “sensor,” “system,” “unit,” or “module” may include a hardware and/or software system that operates to perform one or more functions. For example, a sensor, module, unit, or system may include a computer processor, controller, or other logic-based device that performs operations based on instructions stored on a tangible and non-transitory computer readable storage medium, such as a computer memory. Alternatively, a sensor, module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” “sensors,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

FIGS. 2A and 2B show diagrams of an example probe 200 of a pulse oximetry system (e.g., pulse oximetry system 10) that may be used to measure blood parameters at a nasal site. Specifically, probe 200 may be affixed to a nasal bridge of a patient, as will be described below with regard to FIGS. 3-5. In some aspects, probe 200, may be placed anywhere along the internal carotid artery including additional locations on the head or neck of a patient. FIG. 2A is a cutaway side view of probe 200, and FIG. 2B is a top view.

As shown in FIG. 2A, probe 200 may include a flexible substrate 202 that may give the probe rigidity while conforming to the nasal anatomy of the patient. For example, the thickness of flexible substrate 202 may be such that probe 200 can bend around and maintain contact with the nose of the patient. The flexible substrate 202 may be enveloped, that is, completely surrounded by a soft, cushion-like sleeve 204. For example, sleeve 204 may be comprised of a compressible foam padding wrapped in an opaque, flexible material, such as vinyl. The opaque material may both help prevent light from escaping the probe and help prevent ambient light from being introduced into the probe. The top portion of probe 200 may be covered with an adhesive 205 to enable attachment of the probe to the patient. For example, adhesive 205 may be a transparent, porous, flexible tape.

Probe 200 may include two cushion portions 206a and 206b, one on each end of the length of the probe and connected by a bridge portion 208. As shown, cushion portions 206a and 206b have a thicker amount of sleeve 204 than bridge portion 208 and are somewhat convex. In some examples, cushion portions 206a and 206b may have a more square shape, and in other examples, cushion portions 206a and 206b may have a more rounded shape. Cushion portion 206a houses an emitter unit 210, and cushion portion 206b houses a detector unit 212. In other examples, cushion portion 206a houses the detector unit 212 and cushion portion 206b houses the emitter unit 210. As described with reference to FIG. 1, emitter unit 210 may include multiple light sources, such as LEDs. Similarly, detector unit 212 may include multiple photo detectors. Emitter unit 210 and detector unit 212 may be fastened to flexible substrate 202 with adhesives 214a and 214b, respectively. Adhesives 214a and 214b may be an electrically conductive epoxy adhesive, for example. Cushion portion 206a may include a transparent portion 216a at the position of the emitter unit 210 such that light from emitter unit 210 may pass through cushion portion 206a. Similarly, cushion portion 206b may include a transparent portion 216b at the position of the detector unit 212 such that light may pass through cushion portion 206b to detector unit 212. For example, transparent portions 216a and 216b may be comprised of a transparent, deformable gel. An advantage of the deformable gel is that it may mold to sides of the nasal bridge and act to distribute pressure over a larger area relative to other transparent mediums.

As described with reference to FIG. 1, the probe 200 may be in communication with other components of the pulse oximetry system, such as an emitter drive unit (e.g., emitter drive unit 108), an input amplifier unit (e.g., input amplifier unit 106), a control and processing unit (e.g., control and processing unit 107), a memory (e.g., memory 109), and a display unit (e.g., display unit 114). Such components may be part of the probe 200 or separate components of the pulse oximetry system. As shown in FIG. 2B, probe 200 may include electric wirings 218 to communicate with the other components of the pulse oximetry system.

As also shown in FIG. 2B, probe 200 has a T-shape structure, with opposite T-ends housing the emitter unit 210 and the detector unit 212 and the electric wirings 218 comprising the T-foot. However, it should be noted that the size and shape of probe 200 may vary. In some examples, a single size and shape of probe may be used on all patients. In other examples, the probe may be manufactured at different sizes and configurations to accommodate different types of patients (e.g., neonates, infants, pediatrics, and adults) and to accommodate different nose sizes and shapes as well as other body parts.

FIGS. 3A-3B are diagrams illustrating how the example probe 200 of FIG. 2 may be positioned on a patient in order to obtain PPG signals from blood originating from the internal carotid artery. FIG. 3A is a partial profile view of a human face illustrating how the probe may be positioned on a nose with respect to internal vasculature. FIG. 3B shows a second partial profile view of a human face illustrating the position of the probe in the context of other anatomical features of the face, such as the eyes. As shown in FIGS. 3A and 3B, probe 200 is shaped to be positioned on nose 302 at the root of the nasal bridge 304. As used herein, the nasal bridge may include a saddle-shaped area of the nose that includes the nasal root and the lateral aspects of the nose and may lie between the glabella and the inferior boundary of the nasal bone and extend laterally to the inner canthi. The root of the nasal bridge may include an area of the nose that is the most depressed, superior part of the nose along the nasal ridge. The bridge portion 208 of probe 200 is bent over the root of the nasal bridge 304 such that one cushion portion is positioned on each side of nose 302. While only one cushion portion 206b is illustrated, it can be assumed that cushion portion 206a is positioned on the opposite side of nose 302 directly across from cushion portion 206b such that an optical path is formed between the emitter unit 210 (not shown) housed within cushion portion 206a and the detector unit 212 (not shown) housed within cushion portion 206b.

The nasal cavity contains terminal branches of the internal carotid artery, namely, the anterior ethmoidal artery. Branches of the internal carotid artery, including the anterior ethmoidal artery, are among the last locations of the human body to experience vasoconstriction under physiological stress conditions because the internal carotid artery supplies blood to the brain. Furthermore, copious amounts of blood are supplied to the nasal cavity in order to warm incoming air before it reaches the lungs. Thus, blood flow through the anterior ethmoidal artery is not subject to substantial ANS regulation, making it an advantageous location from which to measure physiological parameters. The anterior ethmoidal artery 306 branches from the ophthalmic artery 308, which stems from the internal carotid artery 310. As shown in FIG. 3A, probe 200 is configured (e.g., shaped) such that cushion portion 206b is positioned over the anterior ethmoidal artery 306 such that detector unit 212 (not shown) receives light transmitted from emitter unit 210 (not shown) after passing through the anterior ethmoidal artery 306, where it may be absorbed by blood contained therein. Thus, probe 200 is positioned to obtain PPG signal data from blood originating from the internal carotid artery 310.

Turning now to FIG. 4, a partial front view of a face of a subject wearing a first embodiment of a nasal pulse oximetry probe 400 is shown. The center of the root of the nasal bridge is indicated at 402. The exact location of the center of the root of the nasal bridge 402 may vary for each patient. In the example of FIG. 4, the center of the root of the nasal bridge 402 is level with the bottom of the eye, as indicated by dashed line 404. However, in other examples, the center of the root of the nasal bridge 402 may be higher than dashed line 404 (e.g., more toward the eyebrows) or lower than dashed line 404 (e.g., more toward the lips). For example, the center of the root of the nasal bridge may lie along a central axis of the eyes (e.g., an axis that passes through the caruncula lacrimalis of each eye).

Bridge portion 408 of the nasal pulse oximetry probe (e.g., probe 200 of FIG. 2A) is shaped to be centered on the center of the root of the nasal bridge 402. In one example, the probe may include visual markers that assist an operator in positioning the probe in the proper position to accurately sense blood flow through the anterior ethmoidal artery. For example, a line may extend horizontally across the bridge portion of the probe, and the operator may position the probe such that the line is substantially aligned with a designated axis of the patient, e.g., the central axis of the eyes.

Cushion portions 406a and 406b are affixed to each side of the nose, for example, by an attachment mechanism such as adhesive. As described above with reference to FIG. 3A, emitter and detector units housed within respective cushion portions, and the cushion portions are shaped to position the emitter and detector units on opposite sides of the nose of the patient at a root of a nasal bridge when the optical probe is worn by the patient. The respective cushion portions are aligned such that an optical path is formed between them. The probe may be positioned with electrical wirings 418 running along the bridge of the nose and onto the forehead in order to minimize patient disturbance by the electrical wirings 418, which may otherwise obstruct the eyes, nose, or mouth, for example. However, the orientation of the electrical wirings does not impact the function of the probe and as such may have different configurations without departing from the scope of this disclosure.

Turning now to FIG. 5, a partial front view of a face of a subject wearing a second embodiment of a nasal pulse oximetry probe 500 is shown. Like components of FIGS. 4 and 5 are numbered similarly (e.g., 408 corresponds to 508). Whereas the nasal pulse oximetry probe of FIG. 4 may be attached to the patient with adhesive, the probe of FIG. 5 additionally or alternatively includes an attachment mechanism in the form of an eyeglass-type frame 510, which rests on ears 516 of the patient. The size and shape of frame 510 may vary. For example, frame 510 may be manufactured at different sizes and configurations to accommodate different types of patients (e.g., neonates, infants, pediatrics, and adults). In particular, the width of frame 510 may vary to accommodate patients with different head widths. The mechanisms for adjusting the probe widths may be part of the frame, which makes one frame suitable for broad range of head widths. Regardless of the size or shape of the frame 510, the frame 510 is configured (e.g., shaped) to position the emitter and detector units on opposite sides of the nose of the patient at a root of a nasal bridge when the optical probe is worn by the patient.

As described with reference to FIG. 4, bridge portion 508 of the nasal pulse oximetry probe is again shaped to be centered on the center of the root of the nasal bridge, indicated at 502, which is again level with the bottom of the eyes, as indicated by dashed line 504. Cushion portions 506a and 506b are pressed against sides of the nose by cushion holders 512a and 512b, respectively. Cushion holders 512a and 512b are attached to frame 510 by wires 514, which may be comprised of a flexible and/or malleable material, such as metals, polymers, polymer blends, etc., to enable the cushion holders to be adjusted with respect to the anatomy of the patient. Cushion holders 512a and 512b may apply pressure to the nasal bridge, which may push surface blood away from the skin of the nasal bridge. Further, the cushion holders may be shaped and sized to position the emitter and detector units at a designated position with respect to the nose e.g., across the root of the nasal bridge. Thus, the probe may more accurately measure light absorption by blood contained within the anterior ethmoidal artery, which is an internal structure, with less interference from surface blood in skin capillaries, for example.

As shown in FIG. 5, electrical wirings 518 again go up the bridge of the nose and onto the forehead of the patient. However, in another example, electrical wirings 518 are housed within frame 510. Furthermore, frame 510 may further house a control and processing unit (e.g., control and processing unit 107 of FIG. 1) for calculating physiological parameters from the signals received from the probe and a radio unit for communicating the physiological parameters to a display device (e.g., display unit 114 of FIG. 1). For example, one or more respiration cycle microfluctuations may be displayed as an arterial blood oxygenation respiratory variability indicator. Information related to physiological parameters including, but not limited to, respiration cycle microfluctuations, may be displayed in a variety of ways on the device and/or on a separate display including, but not limited to, through the use of numeric, visual, or audible indicators.

The amount of light transmitted through a tissue varies according to changes in blood volume at the site. Specifically, the amount of light transmitted through vascular bed decreases during systole (e.g., more light is absorbed) and increases during diastole, resulting in periodic PPG waveforms (e.g., the AC/DC component). Multiple physiological parameters can be extracted from the PPG waveforms measured by a pulse oximetry probe. As an advantage of the copious blood supply to the nasal cavity and flowing through the anterior ethmoidal artery, the resulting PPG data measured by a nasal pulse oximetry probe has a high signal-to-noise ratio. For example, noise may be caused by non-physiological processes such as ambient light and bodily movement. In some aspects the noise may be filtered out from the internal carotid artery PPG, such as the nasal PPG, because these artifact signals remain, in most cases, weak in comparison to the cardiac pulsation. Such artifacts may be filtered using any means generally used. For example, in some aspects PPG signal and individual PPG pulse quality may be classified based on variability in pulse amplitude, pulse shape, interbeat interval or SpO2 and the periods of signal with low signal quality is down weighted or rejected. In another example, PPG signal quality is assessed based on signal autocorrelation or cross-correlation between PPG measured with different wavelengths.

Turning now to FIG. 6, a high-level flow chart of a method 600 for calculating physiological parameters from the PPG signal is shown. Method 600 and the other methods described herein may be performed with a pulse oximetry system, such as pulse oximetry system 10 shown in FIG. 1. More specifically, method 600 and the other methods described herein may be executed by a control and processing unit of the pulse oximetry system (such as control and processing unit 107 shown in FIG. 1) according to instructions stored on a non-transitory memory of the system (e.g., memory 109 shown in FIG. 1) in combination with the various signals received at the control and processing unit from the system components and actuation signals sent from the system controller to the display device (e.g., display device 114 of FIG. 1). The methods included herein will be described with regard to a nasal pulse oximetry probe, although it should be understood that pulse oximetry probes placed on other parts of a body could be used without departing from the scope of the methods.

Method 600 begins at 602 and includes receiving probe output from a nasal pulse oximetry probe or other internal carotid artery probe (e.g., probe 200 of FIG. 2A). As indicated at 604 and described with reference to FIG. 3A, the probe is positioned to measure blood within the anterior ethmoidal artery of a patient. In some examples, a clinician or other user may position the probe based on visual markers present on the probe in order for the probe to measure blood within the anterior ethmoidal artery. Additionally or alternatively, the clinician may scan the positions along the bridge of nose, or other areas of the head and neck, and find a position providing the strongest pulse.

At 606, method 600 includes calculating, that is electronically measuring, one or more physiological parameters from the probe output. For example, physiological parameters may include SpO₂, pulse transit time (PTT), blood circulation at the measurement site, respiration rate, respiration variability, and an indication of overall blood volume. Calculating the physiological parameters may include using algorithms stored in a memory of a control and processing unit (e.g., memory 109 of FIG. 1), including an SpO₂ algorithm (e.g., SpO₂ algorithm 111 of FIG. 1), a PTT algorithm (e.g., PTT algorithm 112 of FIG. 1), a respiration rate algorithm (e.g., respiration rate algorithm 116 of FIG. 1), a blood circulation algorithm (e.g., blood circulation algorithm 110 of FIG. 1), and a hypovolemia algorithm (e.g., hypovolemia algorithm 115 of FIG. 1). Examples of different calculations will be described with regard to FIGS. 7-11.

At 608, method 600 includes storing the physiological parameters (e.g., as calculated at 606) in the memory and/or displaying the parameters via a display device (e.g., display unit 114 of FIG. 1). Physiological parameters may be stored in the memory in order to monitor how the physiological parameters of the patient change over time. For example, if the patient is undergoing surgery, it may be beneficial to monitor for hypovolemia (decreased circulating blood volume) due to bleeding. Furthermore, the physiological parameters may be displayed on the display device so that a care provider may choose an appropriate course of action or make a diagnosis, for example. Following 608, method 600 ends.

FIG. 7 shows an example method 700 for calculating SpO₂ of a patient based on the PPG signal data obtained by a nasal pulse oximetry probe. Method 700 begins at 702 and includes determining the ratio of ratios

$$( R = \frac{{( \frac{AC}{DC} )}_{red}}{{( \frac{AC}{DC} )}_{IR}} ) ,$$

where AC is the valley-to-peak amplitude and DC is the baseline of the light transmission, based on probe output from the nasal pulse oximetry probe. Thus, to determine R, PPG waveforms for two wavelengths of light, one red and one infrared (IR), are recorded. At the red wavelength, Hb absorbs more light and HbO₂ transmits more light. At the IR wavelength, HbO₂ absorbs more light and Hb transmits more light. Thus, the light transmission is higher and the PPG amplitude is correspondingly smaller at the red wavelength (and vice versa at the IR wavelength) when blood has a high oxygen saturation, and light transmission is lower and PPG amplitude larger at the red wavelength (and vice versa at the IR wavelength) when blood has a low oxygen saturation. As can be seen from the above equation for R, a low R value corresponds to a high amount of HbO₂ compared to Hb and thus a high arterial oxygen saturation.

At 704, method 700 includes calculating SpO₂. In some aspects, the relationship between arterial oxygen saturation directly measured from an arterial blood sample and R, as calculated at 702, is determined in a volunteer test in a laboratory setup. The pulse oximetry calibration is then established so that the SpO₂ output at each saturation level is set to show the blood oxygen saturation as closely as possible. The calibration is stored in the pulse oximeter memory, for example, in the form of a lookup table or a polynomial function with R as input and SpO₂ as output. In other aspects, SpO₂ may be calculated based on the ratio of light transmitted at a first wavelength, for example ˜660 nm, that is the pleth signal, to the ratio of light transmitted at a second wavelength, for example ˜940 nm. A series of ratios may be calculated over a plurality of durations or within a single duration to generate a series of SpO₂ indications. While the series of SpO₂ indications may be used for any purpose, in some aspects the series of SpO₂ indications may be used to show a trend in oxygen saturation and/or respiratory variability. For example, the series of SpO₂ indications may be used to electronically measure one or more respiration cycle microfluctuations.

At 706, method 700 includes outputting SpO₂. For example, SpO₂ may be displayed on a display device of the pulse oximeter (e.g., display device 114 of FIG. 1) and/or saved to a memory of the pulse oximeter (e.g., memory 109 of FIG. 1). Following 706, method 700 ends. In some aspects a series of SpO₂ indications may be output as a trend line.

In some aspects, SpO₂ may be used to determine respiration cycle microfluctuations in arterial oxygen saturation. In some aspects, microfluctuations or respiration cycle related variability in arterial oxygen saturation may be a change of less than 10% of the periodic SpO₂ level. For example the SpO₂ may vary by less than about 8%, less than about 5% or less than about 1%. In one example, SpO₂ values are calculated for short epochs of PPG (“SpO₂ instant”) to calculate a series of SpO₂ values.

The series of SpO₂ values (indications) may be analyzed to identify SpO₂ values over a first threshold and/or below a second threshold. A differential value between the values greater than the first threshold and the values less than the second threshold may be calculated to determine one or more respiration cycle microfluctuations in arterial oxygen saturation, where the microfluctuation is the differential value. In some aspects, these values (indications) may indicate maximum and minimums in the series of arterial oxygen saturation indications. In another example, FFT may be applied to the series of SpO₂ values to calculate an amplitude spectrum. The amplitude spectrum may be analyzed to identify the respiration frequency and the related SpO₂ variation amplitude. In some aspects the “SpO₂ instant” in a series of SpO₂ values is calculated twice for each PPG pulse in a time domain by detecting PPG pulse peaks and valleys in series of PPG measurements and calculating SpO₂ separately for rising and falling edges of the pulses. In another aspect the “SpO₂ instant” in a series of SpO₂ values is calculated for time epochs of shorter than or equal to a cardiac interbeat interval with a linear regression based method applied for two PPG channels. The linearity of the processed red and infrared signals allows the use of statistical techniques such as linear regression and linear correlation to fit a best fit straight line to the set of pairs of processed red and infrared data points and to measure the goodness of the straight line fit to these data points. The result of this analysis is a linear regression slope and a goodness of fit correlation coefficient. The correlation coefficient is a measure of the linearity of the input data points and, if less than a predetermined threshold, it indicates that a distorted signal has been received from the probe. The computed linear regression slope may be converted to a ratio of ratios, R, which is used in an empirical calibration formula to compute the SpO₂ value. The minimum size of the data set required for high confidence calculations using this apparatus is significantly smaller than the pulse period and permits faster response to changing input data. The respiration cycle microfluctuations may be converted to an arterial blood oxygenation respiratory variability indicator. Such an indicator may be an output to a display such as display unit 114 and may be numeric, visual, or audible indicator.

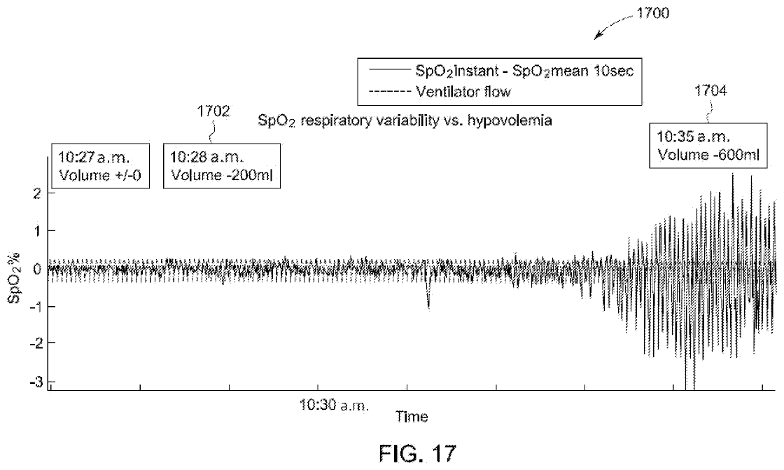

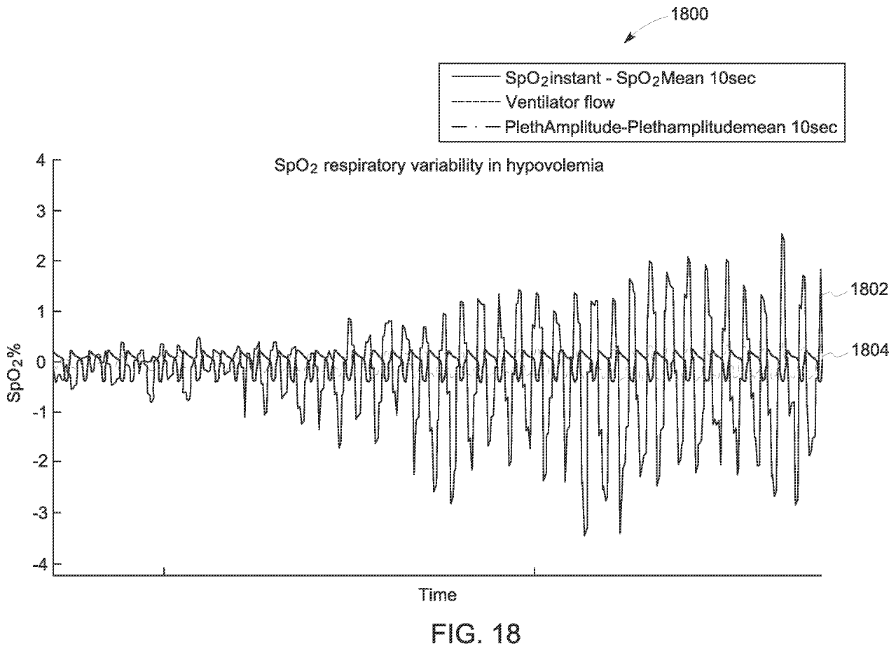

In some aspects the series of SpO₂ indications may be filtered to remove artifacts such as those generated by interference such as movement. Such artifacts may be filtered using any means generally used. In some aspects, SpO₂ indications with the highest levels of variability may be removed from any further calculations. For example, SpO₂ indications over a duration may be sorted and at least one of the largest and one of the smallest values may be removed from each series. In other examples, SpO₂ indications that exceed a threshold percentage difference between a maximum SpO₂ indication and a minimum SpO₂ indication may be removed. In additional examples, SpO₂ variation cycles that do not match the respiration rate of the patient are removed. As an example, a respiration rate provided by a ventilator, respiratory gas module, or other respiration rate measurement may be used for this purpose or the acceptance limits may be set by expected maximum and minimum respiration rates of the patient. In another example, artifacts may be filtered by only using PPG pulses with periods, amplitudes, or signal quality that meet a threshold. In one aspect, as shown in FIG. 17, the series of SpO₂ indications may be high pass filtered to remove the SpO₂ baseline and to filter out SpO₂ variability that is of lower frequency than the respiration rate. The high pass filtered SpO₂ indications may then be plotted as a visual trend to show the SpO₂ respiratory variation.

In some aspects, SpO₂ output may be used to provide an estimation of intravascular volume, or as an indicator of hypervolemia. As shown in FIG. 17, shortly after blood volume begins to decrease at 10:28 am as shown at 1702, there is an increase in SpO₂ variability with the variability increasing as bleeding continues. As hypovolemia increases so does the SpO₂ variability with significant variability shown at 10:35 am at 1704. This variability may be detected according to the methods described herein. As shown in FIG. 18, during hypovolemia there can be significantly greater SpO₂ variability as shown by the solid line at 1802 than pleth variability as shown by the dash-dot line at 1804. Thus, hypovolemia may be indicated when the SpO₂ variability amplitude is greater than a threshold amount for a duration. In some aspects, the threshold amount may be a function of the sigmoid shape of the oxygen-hemoglobin dissociation curve.

Thus, at least in some aspects, the pulse oximetry system described herein may be used to calculate instantaneous SpO₂ values (also referred to herein as SpO₂ instant measurements or SpO₂ indications), a series of which may be plotted, filtered, normalized, and/or otherwise represented and analyzed in order to identify whether one or more respiration cycle microfluctuations are present, which may indicate arterial blood oxygenation respiratory variability. Each instantaneous SpO₂ value may be determined based on light transmission changes at a first wavelength and a second wavelength (e.g., PPG waveforms), with the light transmission changes determined over a first, shorter period of time and/or at a first, higher frequency. These instantaneous SpO₂ measurements may be differentiated from a conventional SpO₂ value that may be generated and output to track patient blood oxygen saturation. Conventional SpO₂ values may be determined based on light transmission changes at the first wavelength and second wavelength, but over a second, longer duration and/or at a second, lower frequency. For example, the instantaneous SpO₂ measurements may be determined from light transmission signals (e.g., PPG waveforms) over a first duration that is less than a respiration period, such as a first duration that is equal to or less than a cardiac interbeat interval, while the conventional SpO₂ measurement may be determined from light transmission signals over a second duration that is equal to or greater than a respiration period. Further, the series of instantaneous SpO₂ values may be filtered or normalized differently than the conventional SpO₂ values. For example, the series of instantaneous SpO₂ values may be mean filtered, such that a mean SpO₂ value (representing the mean of the prior N instantaneous SpO₂ values) is subtracted out of each instantaneous SpO₂ value, which may boost the variability signal. In contrast, the conventional SpO₂ value may be a mean value and/or or may not be filtered or normalized.

In other aspects, SpO₂ variability may be converted to an estimated PaO₂ variability by using the expected oxygen-hemoglobin dissociation curve. Respiration cycle related PaO₂ variation may be related to varying pulmonary shunt fractions. PaO₂ oscillations may be used as an indication of cyclical recruitment of atelectasis. In healthy lungs, respiration cycle related PaO₂ variation/microfluctuations may be an indication of hypovolemia related cyclical redistribution of pulmonary perfusion. Because of the sigmoid shape of the oxygen-hemoglobin dissociation curve, the same level of hypovolemia causes lower SpO₂ respiratory variation at high SpO₂ levels than the variation measured when patient has a low SpO₂ level. The hypovolemia indication may also be based on a combination of respiration related arterial blood oxygenation (SpO₂ or estimated PaO₂) and plethysmograph amplitude variability. In some aspects, SpO₂ variability may additionally be used to access patient fluid responsiveness, that is, the ability of the heart to increase stroke volume in response to an increase in fluid.

This respiration cycle related oxygenation variability is generally filtered out from the SpO₂ measured with conventional pulse oximetry using peripheral sensor sites (e.g. finger or toe) but is visible in the SpO₂ measured from the signal originating from the carotid artery e.g. with a probe attached on the nasal root. Additionally, conventional pulse oximeters use data epochs for SpO₂ calculation that are too long and respiration related variability is filtered out.

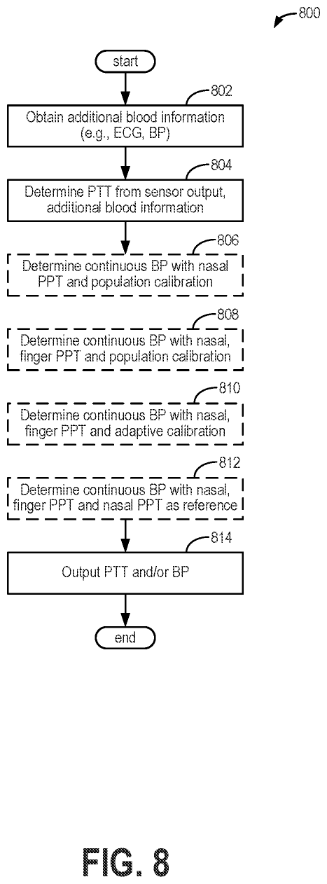

Turning now to FIG. 8, a method 800 is illustrated for calculating pulse transit time (PTT) of a patient from PPG signal data obtained by a nasal pulse oximetry probe (e.g., probe 200 of FIG. 2A) positioned to measure blood originating from the internal carotid artery. PTT is defined as the time it takes a pulse pressure waveform to propagate through a length of the arterial tree, typically from the aorta to a peripheral arterial site. PTT is usually measured, in part, with information from an electrocardiogram (ECG). As such, a first time point is the ECG R-peak time, and a second time point is the beginning of the systolic increase of blood volume, e.g., when the PPG waveform begins to rise, at the peripheral arterial site, typically the measurement site on which the PPG probe is positioned (e.g., the nasal bridge or finger). PTT can be related to blood pressure (BP), which thus enables continuous non-invasive BP measurement. Specifically, PTT can be used to determine a pulse wave velocity, which is related to vessel stiffness. The greater the stiffness, the higher the pulse wave velocity. Artery stiffness is modulated by factors such as age, atherosclerosis, and BP. At high BPs, artery walls are tense and hard, making the pulse wave travel faster (e.g., PTT is reduced). At low BPs, the artery walls have less tension, making the pulse wave travel slower (e.g., PTT is increased).

Referring to FIG. 8, method 800 for blood pressure determination begins at 802 and includes measuring electrical activity of the patient's heart over a period of time using electrodes placed on the chest. The resulting data may be output as an ECG, for example. Simultaneously, PPG signals are measured at the nasal and/or finger site (as described above with respect to FIG. 6).

At 804, method 800 includes determining a nasal PTT and/or a finger PTT from the ECG R-peak and a specific time point in the PPG systolic rise at the nasal and/or finger sites. For example, a control and processing unit (e.g., control and processing unit 107 of FIG. 1) may use a PTT algorithm (e.g., PTT algorithm 112 of FIG. 1), in which an ECG processing algorithm detects the ECG R-peak by detecting the maximum of the R-peak and a PPG processing algorithm detects specific time points during the systolic rise. For example, the points of maximum derivative (e.g., the time at which the first derivative of the PPG waveform is maximal) of the nasal and finger PPG waveforms may be detected. Therefore, two PTTs, one for the nasal PPG and the other for the finger PPG, are determined. The R-wave peak time corresponds to when the heart ventricle contracts, and the PPG maximum derivative time corresponds to when blood ejected from the heart by the ventricle contraction reaches the measurement site. Of note, there is a ˜250 ms delay from the R-peak for the pulse wave to reach a finger. However, this delay does not occur at the nasal site or other sites on the head or neck because the distance from heart is shorter and because vasoconstriction in the artery due to ANS regulation is almost non-existent in the (high priority) intra-cranial blood flow. Therefore, the stiffness of the carotid artery, and thereby, the pulse wave velocity, is influenced only by blood pressure, whereas at peripheral sites, the pulse velocity is also affected by other (confounding) factors, such as sympathetic activation (vasoconstriction) or blood volume changes. Consequently, the PTT determined by a nasal pulse oximeter most likely reflects the effects of BP changes more profoundly than PTT determined by a pulse oximeter measuring blood at the finger or other extremity. Further, it has been shown that the pleth signal from an internal carotid artery site may be as much as 10 times greater than the signal obtained from a finger. On the other hand, because the distance of the finger from the heart is longer, blood pressure changes the absolute PTT value measured at the finger more than at the nasal site.

The PTT(s) may be used in one or more algorithms to determine continuous blood pressure (BP). There are several ways to use PTT to measure BP. In a first example, as indicated at 806, continuous BP may be determined based on nasal PTT and a population calibration. As described above, the PTT is determined from the ECG R-peak to the systolic rise (maximum derivative) in the nasal PPG. As discussed above, the benefit of this method is that the PTT is least affected by confounding ANS regulation, hypovolemia, hypothermia, and atherosclerosis of peripheral arteries, for example. The BP may be measured by first determining the relationship between PTT and BP in a large patient population. In these calibration runs, the BP is determined, for example, invasively or using non-invasive blood pressure (NIBP) measurement, after which the functional relationship between BP and PTT may be established. This relationship may then be used on patients of the same population characteristics. For example, a calibration equation may be calculated that is different for different patient populations, such as children and adults.

In another example for a continuous BP measurement, as indicated at 808, the continuous BP may be determined based on the PTTs determined for both the nasal and finger sites and the population calibration described above. The PTTs may be calculated using the ECG R-peak time point as a reference. A benefit of this embodiment is that a BP change is larger in absolute value for the finger PTT. As discussed above, however, the finger PTT may be confounded by ANS regulation or other reasons. In this situation, the nasal PTT may be beneficially used to individualize the relationship between the two measured PTTs and the patient's individual BP. As a result, a large part of the patient-to-patient variability in the particular patient population may be removed.

As a further example, as indicated at 810, continuous blood pressure may be determined based on the finger and nasal PTTs and an adaptive calibration. For example, a two point calibration of the measured PTT values against a NIBP measurement at two different levels of BP may be calculated for one patient. The PTT and BP values may be obtained close in time so that confounding factors are likely constant during the two-point calibration interval. The assumption is valid for such confounding factors as hypovolemia, hypothermia, and blood vessel atherosclerosis. In one example, a linear model between BP and the nasal and finger PTT (PTT1 and PTT2, respectively) may be assumed, according to equation A:

$$( \begin{matrix}BP \\ C\end{matrix} ) = ( \begin{matrix}c 11 & c 12 \\ c 21 & c 22\end{matrix} ) ( \begin{matrix}PTT 1 \\ PTT 2\end{matrix} )$$

The above equation has four unknown coefficients, c11, c12, c21, and c22. In a first step of calibration, the coefficients c11 and c12 are determined so that the correct BP values are obtained at the two NIBP levels. The output parameter C may be used for detecting changed confounding factor situations, e.g., those that may occur due to blood volume changes or temperature changes. At normal patient conditions, the value of C may be set to be a fixed constant at both BP calibration levels. The coefficients c21 and c22 may then be determined in the calibration phase at the two levels of BP. Multiple point calibration may be used to improve the calibration process. In this case, the coefficients c11, c12, c21, and c22 are determined using a statistical optimization analysis, such as linear least square optimization or linear regression. After calibration, the equation Eq. A is used to calculate BP from the measured PTT values, e.g., PTT2 for the finger and PTT1 for the nasal site. In one example, the constant C may be used to alert about a changed patient condition due to reasons other than BP. When a change in C is observed, the coefficients c11 and c22 may be determined again by requesting NIBP calibration from a user. The NIBP measurement is activated at least at one time point and at one BP level, and the corrections for the coefficients c11 and c12 (as well as c21 and c22) are determined.

The above adaptive method may be improved by using a multiple point original calibration with a non-linear relationship between PTT1 and PTT2 and a large patient population with different (mixed) levels of confounding factors. For example, BP may be calculated as BP=f(PTT1, PTT2, + other variables such as HR), in which f is the non-linear function for BP having PTTs and other physiological parameters as independent variables.

A further example for continuous BP measurement using PTT measurement is indicated at 812, where the continuous BP is determined based on the finger and nasal PTTs using the nasal PTT as a reference (rather than the ECG R-peak as described above). Since PTT to the nasal root is very short (e.g., relative to the finger PTT) at some tens of milliseconds, the nasal plethysmogram may be used as a reference time point for the finger PTT. In this case, the ECG waveform is not needed. The finger PTT is then calculated from the nasal pleth systolic rise to the finger pleth systolic rise, for example, both determined as the maximum derivative of the pleth waveform. This PTT may then be calibrated against NIBP BP determinations using standard methods.

At 814, method 800 includes outputting PTT and/or BP. For example, PTT and/or BP may be displayed on a display device of the pulse oximeter (e.g., display device 114 of FIG. 1) and/or saved to a memory of the pulse oximeter (e.g., memory 109 of FIG. 1).

Following 814, method 800 ends.

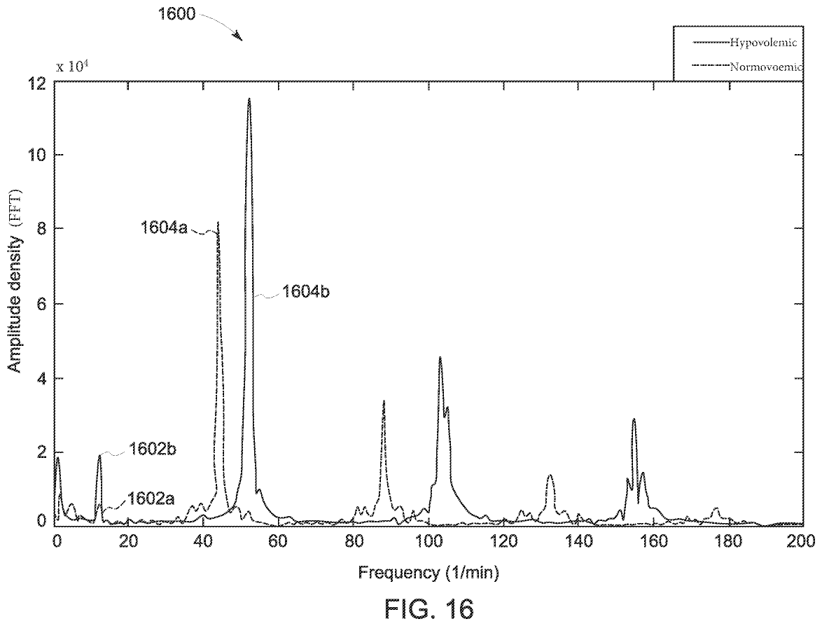

Turning now to FIG. 9, a method 900 for determining respiration rate using a nasal pulse oximetry probe (e.g., probe 200 of FIG. 2A) positioned to measure blood originating from the internal carotid artery is shown. As a patient breathes, changes in intrathoracic pressure throughout a respiratory cycle cause respiration-related variations in the PPG waveform, including baseline modulation (the DC component, due to changes in venous return), amplitude modulation (the AC component, due to a decreased left ventricular stroke volume during inspiration), and frequency modulation (due to the fact that heart rate increases during inspiration and decreases during expiration). In standard pulse oximetry, these variations are often filtered out to determine SpO₂. However, using the systems and methods described herein, the inventors have determined that the waveform modulations may be used to determine the respiration rate of a subject from the PPG signal. Notably, respiration-related variation is stronger in PPG signals measured at the root of the nasal bridge than measured at a finger or ear, resulting in better signal-to-noise separation, which may improve the accuracy of respiration rate calculations. The inventors herein have recognized that another parameter, SpO₂, experiences respiration-related variation as shown in FIG. 18. As shown in FIGS. 14 and 16, microfluctuations may not be detectible or may be difficult to detect in the PPG signal measured at a finger, for example, but are detectible in the PPG signal measured at the root of the nasal bridge or other internal carotid artery location. Thus, the microfluctuations in SpO₂ measured by a nasal pulse oximeter or other internal carotid artery pulse oximeter may also be used to calculate respiration rate, as described in further detail below.

Method 900 begins at 902 and includes calculating a first respiration rate based on probe output in a time domain. PPG data is collected over time, resulting in the PPG waveform. A control and processing unit (such as control and processing unit 107 of FIG. 1) may extract respiration-related variations in the PPG waveform and use an algorithm (e.g., respiration rate algorithm 116 of FIG. 1) to calculate respiration rate based on these variations. For example, the control and processing unit may extract one or more of the baseline modulation, amplitude modulation, and frequency modulation of the PPG waveform to calculate the respiration rate. Respiration rate may be calculated from probe data obtained over a first predetermined duration (e.g., 45 seconds), for example. Further, the respiration rate may be recalculated at a predetermined frequency (e.g., every 5 seconds). In some aspects, the duration may be relative to a heartbeat or cardiac interbeat interval. In some aspects, the cardiac interbeat interval is less than half of a respiration period where the respiration period is the amount of time elapsed for each inspiration and expiration cycle.

At 904, method 900 includes calculating a second respiration rate based on microfluctuations in SpO₂. Microfluctuations or respiration cycle related variability in arterial oxygen saturation may be calculated based on the differential between SpO₂ values over a first threshold and SpO₂ values below a second threshold. In some aspects a microfluctuation may be a change of less than 10% of the SpO₂ level. For example the SpO₂ may vary by less than 8%, less than 5% or less than 1%. In the previous example, data from a PPG waveform measured at one wavelength (typically infrared) may be used to determine respiration rate. In another example, the complete two waveform data, e.g., the red and infrared PPGs, may be used to determine respiration rate. The respiration rate may be determined from the ratio of ratios, R, by first calculating R separately for systolic and diastolic phases of the two PPGs, with R calculations repeated pulse by pulse (e.g., for each heartbeat or interbeat interval) or multiple times per interval, as described further below with reference to FIG. 13.

For example, in some aspects the respiration rate may be calculated from a series of SpO₂ values where each of the SpO₂ values is determined based on PPG measured over a duration, for example over a duration less than or equal to a cardiac interbeat interval. For example, the duration may be less than ½ of the respiration period to ensure reliable SpO₂ respiration variability measurement. For example, assuming a heart rate of 60 beats per minute, an interbeat interval is 1 second and a respiration rate may be 15/min yielding a respiration period of 4 seconds. The duration over which PPG data used for the SpO₂ calculation is collected may vary adaptively based on the patient heart rate and respiration rate or may be of a constant duration of a length which provides 2 or more SpO₂ values for a normal range of respiration cycles, e.g. the duration may be fixed as 1 second. In other aspects, the duration over which a series of SpO₂ values are calculated is greater than or equal to a respiration period, for example it may be equal to two or more respiration periods. A trend in the series of SpO₂ calculations and respiration rate may be indicative of specific physiological parameters. For example, during early stages of deterioration, a patient's SpO₂ may appear to be in the normal range, but the respiration rate may increase in response to inadequate gas exchange. Thus the identification of microfluctuations and calculation of respiration rate over a short duration as described above provides an opportunity for timely intervention.

Turning briefly to FIG. 12, an example graph 1200 illustrating how R may be determined separately for systole and diastole is shown. As shown, R may be determined by plotting (AC/DC)_{red} (Y-axis) against (AC/DC)_{IR} (X-axis), with the resulting graph forming a loop. A top portion of the loop, indicated at 1202, comprises measurements taken during systole (the phase of plethysmogram during which the signal increases), and a bottom portion of the loop, indicated at 1204, comprises measurements taken during diastole (the phase of the plethysmogram during which the signal decreases). The slope of the top of the loop 1202 is equal to R during systole (R_{systole}), while the slope of the bottom of the loop 1204 is equal to R during diastole (R_{diastole}). A difference between R_{systole} and R_{diastole} is due to respiration-related variation.

Continuing to FIG. 13, an example graph 1300 is shown illustrating respiration rate (plot 1302), wherein a ratio of ratio of ratios (Y-axis), defined as the ratio of R_{systole} and R_{diastole}, is plotted versus heartbeat (X-axis). The ratio of ratio of ratios gives the respiration signal, which may be used to determine the second respiration rate using a respiration rate algorithm (e.g., respiration rate algorithm 116 of FIG. 1).

Returning to FIG. 9, at 906, method 900 includes outputting a calculated respiration rate based on one or more of the first respiration rate and the second respiration rate. In one example, the first and the second respiration rates are averaged and output as the calculated respiration rate. In another example, one respiration rate (either the first or the second) is output responsive to the magnitude of the difference between the first and second respiration rate being within a threshold. Following 906, method 900 ends.

By calculating the respiration rate in more than one way, the accuracy of the resulting value may be improved compared with calculating the respiration rate one way. Further, the respiration rate may be more accurately determined using a nasal pulse oximetry probe positioned at the root of the nasal bridge compared with a pulse oximetry probe positioned at an extremity due to stronger respiration-related variation observed at the nasal site.

Changes in respiration-related variation can be used to determine additional physiological parameters. Turning now to FIG. 10, a method 1000 for determining if a patient is hypovolemic using a nasal pulse oximetry probe (e.g., probe 200 of FIG. 2A) positioned to measure blood originating from the internal carotid artery is shown. Hypovolemia is defined as a state of decreased blood volume, which may be caused by blood loss or sodium depletion, for example.

Method 1000 begins at 1002 and includes transforming probe output to a frequency domain. For example, the probe output obtained over a duration (e.g., 60 seconds) may be analyzed in the frequency domain using a fast Fourier transform (FFT). The resulting spectrum shows the PPG signal plotted as amplitude density against frequency.

Turning briefly to FIG. 14, a graph 1400 of example spectra of transformed PPG signal data received from a pulse oximeter placed along the internal carotid artery is shown. The X-axis of graph 1400 represents frequency, and the Y-axis represents amplitude density. An example spectrum of transformed PPG signal data measured from a normovolemic patient is represented by a dashed line, and an example spectrum of transformed PPG signal data measured from a hypovolemic patient is represented by a solid line. A first peak indicated at 1402a (normovolemia) and 1402b (hypovolemia) occurs in each spectrum at a respiratory frequency (˜10/min), and a second peak indicated at 1404a (normovolemia) and 1404b (hypovolemia) occurs in each spectrum at a cardiac frequency (which typically ranges from 40-90/min for a resting adult). Each peak also has corresponding higher frequency harmonics. Similarly, in FIG. 16, a graph 1600 of transformed PPG signal data received from a pulse oximeter placed on a finger is shown. A first peak indicated at 1602a (normovolemia) and 1602b (hypovolemia) occurs in each spectrum at a respiratory frequency (˜10/min), and a second peak indicated at 1604a (normovolemia) and 1604b (hypovolemia) occurs in each spectrum at a cardiac frequency (which typically ranges from 40-90/min for a resting adult). As shown in a comparison of FIG. 14 and FIG. 16, the signal of the first peak is much stronger when measured from the nasal root than from a finger. The strength of the signal becomes increasingly important during conditions of physiological stress such as hypotension, hypothermia, and volumetric shock, all of which may result in vasoconstriction, further decreasing the reliability of a signal taken from the extremities.

During hypovolemia, there is an increase in respiratory-induced variation due to the greater impact positive pressure has on a lower blood volume. Therefore, at the respiratory frequency, the first peak 1402b (or 1602b) of the spectrum corresponding to the hypovolemic patient (solid line) has a greater amplitude density than the first peak 1402a (or 1602a) of the spectrum corresponding to the normovolemic patient (dashed line). Thus, it may be possible to determine hypovolemia in a patient based on the amplitude density at the respiratory frequency (e.g., the respiration-related variation), as described below. In an embodiment, the ratio of the spectral amplitudes at the respiratory frequency and cardiac frequency is determined. This ratio is high for a hypovolemic patient and low for a normo- or hypervolemic patient. As shown in the comparison of FIGS. 14 and 16, the respiratory frequency peak of transformed PPG signal data acquired with a finger pulse oximeter is weaker and less sensitive to volemia status than the respiratory frequency peak of transformed PPG signal data acquired with a carotid artery pulse oximeter, whether that carotid artery pulse oximeter is a nasal pulse oximeter, or a pulse oximeter placed at other locations on the head and neck. Thus, it may be advantageous to determine volemia status with a carotid artery pulse oximeter such as the nasal pulse oximeter.

Returning to FIG. 10, at 1004, method 1000 includes determining the amplitude density of the respiratory frequency signal (e.g., peaks 1402a and 1402b in FIG. 14). The amplitude density of the respiratory frequency signal gives an indication of the respiration-related variation.

At 1006, the method includes indicating hypovolemia when the amplitude density is greater than a threshold. In another example, hypovolemia is indicated when the amplitude density of the respiratory frequency signal increases by a threshold amount as shown in FIG. 17. In still another example, hypovolemia is indicated when the ratio of the respiratory and cardiac spectral peaks is greater than a threshold. An indication of hypovolemia may be output on a display device (e.g., display device 114 of FIG. 1), for example. In some aspects, an indication of hypovolemia may be output as an alarm, for example a numeric, visual or audible alarm. Following 1006, method 1000 ends.

FIG. 11 shows a method 1100 for determining blood circulation at a measurement site (e.g., the root of the nasal bridge) using a nasal pulse oximetry probe (e.g., probe 200 of FIG. 2A). Blood circulation may be determined by changing the volume of venous blood returned to the heart, as described below. The change in the blood volume returned to the heart may be determined in various ways, for example, by performing a passive leg raising procedure, changing the patient position (supine, sitting up-right positioning), or by changing intra-thoracic pressure, if the patient is on a ventilator. As will be described in more detail below, the change in blood volume returned may be determined using a change in positive end-expiratory pressure (PEEP). Because the measurement site is supplied by the internal carotid artery, blood circulation at the measurement site also gives an indication of blood flow to the brain. Such an indication cannot be obtained by measuring blood circulation at an extremity, such as a finger or ear.

Method 1100 begins at 1102 and includes obtaining a first probe output with a first amount of positive end-expiratory pressure (PEEP). The probe output may be the DC level of the total light transmission. It is further noted that only the infrared output may be used. PEEP refers to a pressure in the lungs that exists at the end of expiration. Extrinsic PEEP may be applied with a ventilator, and an amount of PEEP may be controlled by settings of the ventilator. PEEP may contribute to decreased venous return, which results in increased blood accumulation, as measured by the pulse oximetry probe at the measurement site.

At 1104, method 1100 includes obtaining a second probe output with a second amount of PEEP. For example, the second amount of PEEP may be higher than the first amount of PEEP, resulting in further blood accumulation. Alternatively, the second amount of PEEP may be less than the first amount of PEEP, resulting in reduced blood accumulation compared with the first amount of PEEP.

Turning briefly to FIG. 15, a graph 1500 is shown illustrating an example of PPG signal data acquired by a nasal pulse oximeter during different amounts of PEEP. The X-axis represents time, with time increasing from left to right, and the Y-axis represents the intensity of transmitted light, with intensity increasing from bottom to top. The red output is shown at plot 1502, and the infrared output is shown at plot 1504. During time ranges t1 and t3, a first, lower amount of PEEP is applied (e.g., 5 cm H₂O). During time range t2, a second, higher amount of PEEP is applied (e.g., 12 cm H₂O). During time range t2, the intensity of transmitted light in the red channel (plot 1502) decreases compared to time range t1 due to further blood accumulation, as the second, higher amount of PEEP further blocks venous return to the heart. There is also a small decrease in the intensity of transmitted light in the infrared channel (plot 1504) during time range t2. The relative changes depend on the overall (venous and arterial) saturation of blood at the site. During time range t3, the intensity of transmitted light in the red channel (plot 1502) increases compared to t2 as the amount of PEEP is reduced to the first, lower amount of PEEP.

Returning to FIG. 11, at 1106, method 1100 includes determining blood circulation based on a difference between the first probe output and the second probe output. For example, a control and processing unit (such as control and processing unit 107 of FIG. 1) in communication with the probe may use the difference between the first probe output (the DC level of the infrared PPG at the first amount of PEEP) and the second probe output (the DC level of the same infrared PPG at the second amount of PEEP) and an algorithm (e.g., blood circulation algorithm 110 of FIG. 1) to determine the blood circulation. Whereas blood flow in the extremities is subject to regulation by the ANS (such as vasoconstriction), which may block the effects of PEEP, blood flow at the root of the nasal bridge is not. As such, the blood circulation algorithm for the nasal pulse oximeter may include less filtering than, for example, a blood circulation algorithm for a finger pulse oximeter. Further, the control and processing unit may use the first probe output and the second probe output to determine a difference in respiration-related variation for the first amount of PEEP and the second amount of PEEP. For example, the difference of the DC levels at the nasal root (or other blood volume indicator signals, such as PPG pulse amplitude variations at the nasal root) at the two different amounts of PEEP may be used to create new parameters that show hypovolemia. These may be used to create more reliable measures of hypovolemia than is possible at one amount of PEEP. Following 1106, method 1100 ends.

Thus, the systems and methods described herein provide for determining various physiological parameters of a patient, including SpO₂, pulse transit time, respiration rate, hypovolemia, cyclical recruitment of atelectasis and intracranial blood circulation, from an output of a nasal pulse oximetry probe configured to measure blood flowing through the anterior ethmoidal artery. In some examples, in addition to determining the physiological parameters using the output from the nasal pulse oximetry probe, an output from a second pulse oximetry probe located at an extremity of the patient, such as a finger or ear, may also be used. Physiological parameters calculated from the output of the nasal pulse oximetry probe may be compared to physiological parameters calculated from the output of the second pulse oximetry probe to determine a state of the autonomic nervous system, for example. For example, if a pulse wave is detected at the nasal site but not at the extremity (or if the pulse wave is diminished at the extremity), it may be determined that the ANS is regulating blood flow to the extremities. Such a comparison is enabled by the fact that blood vessels in the extremities are subject to vasoconstriction and the anterior ethmoidal artery is not. As a result, the physiological parameter calculations described herein (e.g., with respect to FIGS. 6-11) may be performed accurately even when a patient is undergoing vasoconstriction, such as when the patient is hypothermic. In another example, if a physiological parameter calculated from each probe output differs by more than a threshold, the physiological parameter may be re-measured. In still another example, the calculations from each probe output may be averaged.

The technical effect of measuring the blood oxygen saturation using a nasal pulse oximetry probe is a more accurate measurement that is not confounded by vasoconstriction.

An example provides a system for an optical probe, comprising a light emitter and a light detector each coupled to a substrate, the light emitter and light detector configured to measure blood originating from an internal carotid artery of a patient; and an attachment mechanism configured to couple the optical probe to a nose of the patient, the light emitter and light detector positioned to be on opposite sides of the nose of the patient at a root of a nasal bridge when the optical probe is worn by the patient. In examples, the optical probe is configured to measure arterial oxygen saturation at the root of the nasal bridge.

The optical probe may further comprise a first soft cushion-like structure that encompasses the light emitter and a second soft cushion-like structure that encompasses the light detector, each cushion-like structure conforming to the nose of the patient, wherein each cushion-like structure is deformable. In an example, each cushion-like structure includes deformable gel. In one example, the first cushion-like structure covers the light detector and the second cushion-like structure covers the light emitter, each cushion-like structure being at least partially transparent such that light from the light emitter is configured to pass through the first cushion-like structure and through the second cushion-like structure to the light detector.

The attachment mechanism may include a pressure element configured to block surface blood circulation of skin of the patient at the root of the nasal bridge. In examples, the attachment mechanism is an eyeglass-type frame.

The light emitter may include a light emitting diode (LED) configured to emit light having a wavelength of between 760 and 950 nm. In an example, the light emitter includes a first LED having an emission wavelength in a range between 620 and 690 nm and a second LED having an emission wavelength in a range between 760 nm and 950 nm.

The substrate may comprise a T-shape with opposite T-ends of the substrate housing the light detector and light emitter, respectively, and a T-foot of the substrate carrying electrical wirings from the optical probe to a measurement unit along the nasal bridge of the nose.

The optical probe may further comprise a control and processing unit that processes signals from the light detector and calculates physiological parameters, the control and processing unit communicatively coupled to a display unit. In an example wherein the attachment mechanism is an eyeglass-type frame, and the control and processing unit may be coupled to the eyeglass-type frame.

Another example system for an optical probe comprises a light emitter and a light detector shaped to be attached on a nasal root of a patient and configured to measure light transmission through blood originating from an internal carotid artery of the patient and output a light transmission signal; and a control and processing unit including instructions to extract a respiration-related variation from a photoplethysmogram obtained from the light transmission signal. The control and processing unit may be configured to extract a respiration rate and/or an amplitude of the respiration-related variation. Further, the control and processing unit may be configured to detect hypovolemia from the respiration-related variation. Further still, the control and processing unit may be configured to measure the respiration-related variation at two physiological conditions that reflect different venous blood return volumes to a heart of the patient, for which a difference of the respiration-related variation is determined.

Another example of a system for an optical probe comprises a light emitter and a light detector shaped to be attached on a nasal root of a patient and configured to measure light transmission through blood originating from an internal carotid artery of the patient and output a light transmission signal; and a control and processing unit having instructions stored in memory or hardware configured to determine microfluctuations in arterial oxygen saturation based on the light transmission signal obtained over a duration. The control and processing unit may be configured to perform a first arterial oxygen saturation calculation for systole and a second arterial oxygen saturation calculation for diastole and determine a variability value, i.e. respiration cycle microfluctuation, wherein the variability value is a difference between the first arterial oxygen saturation indication and the second arterial oxygen saturation indication. In some aspects, the variability value may be a representative value of the plurality of variability values measured over multiple respiration periods. In some aspects the variability value or respiration cycle microfluctuation may be filtered as described above such that a representative value(s) may be calculated. That is, the median value in a series of differences between the first arterial oxygen indication and the second arterial oxygen saturation indication or calculations based on arterial oxygen saturation indications with pre-determined periods, amplitudes, and/or signal quality. In an example, the control and processing unit may be configured to use the microfluctuations in arterial oxygen saturation to calculate a respiration rate of the patient. In another example, the control and processing unit may be configured to use the microfluctuations in arterial oxygen saturation to determine a respiration-related variation.

In another representation, a method comprises receiving probe output from a nasal pulse oximetry probe attached to a subject at a measurement site and calculating one or more physiological parameters from the probe output. In examples, the measurement site is a root of a nasal bridge. In one example, calculating the one or more physiological parameters includes calculating SpO₂. In examples, SpO₂ may be calculated from a ratio of ratios (R), wherein the ratio of ratios is determined from the probe output measured at two different wavelengths of light. In one example, SpO₂ may be calculated using R and an empirical calibration that relates arterial oxygen saturation values directly measured from arterial blood samples and corresponding R values.

In another example, calculating the one or more physiological parameters includes calculating pulse transit time (PTT). For example, calculating PTT may comprise determining a first time from an electrocardiogram (ECG), wherein the first time corresponds to an R-peak time, and a second time from the probe output, wherein the second time corresponds to a time at which blood volume increases at the measurement site. Further, in some examples, PTT may be used to calculate continuous blood pressure (BP). For example, continuous BP may be calculated using PTT and a population calibration, wherein the population calibration includes measuring PTT and measuring BP for a plurality of subjects and determining a functional relationship between BP and PTT. In another example, continuous BP may be calculated using PTT and an adaptive calibration. The adaptive calibration may include measuring PTT and BP at two or more BP levels and determining a relationship between BP and PTT.

In an example, calculating the one or more physiological parameters includes calculating blood circulation at the measurement site. In examples, calculating blood circulation comprises obtaining a first probe output at a first amount of positive end-expiratory pressure (PEEP) and obtaining a second probe output at a second amount of PEEP. In one example, calculating blood circulation further comprises determining a difference in respiration-related variation for the first amount of PEEP and the second amount of PEEP from the first probe output and the second probe output, respectively, wherein the difference in respiration-related variation gives an indication of total blood volume.

In another example, calculating the one or more physiological parameters includes calculating respiration rate. In one example, calculating respiration rate comprises obtaining probe output over a duration and identifying a variation in the probe output over the duration, wherein the frequency of the variation is used to calculate respiration rate. The variation in the probe output may include one or more of a baseline modulation, an amplitude modulation, and a frequency modulation. In another example, calculating respiration rate comprises calculating microfluctuations in SpO₂ for each heartbeat of the subject, wherein the microfluctuations in SpO₂ are determined by calculating a ratio of a first ratio of ratios (R) for a systolic phase and a second R for a diastolic phase of each heartbeat.

In another example, calculating the one or more physiological parameters includes calculating respiration variability. In one example, calculating respiration related variation comprises obtaining probe output over a duration and identifying a variation in the probe output over the duration. The variation in the probe output may include one or more of SpO₂ microfluctuations, PPG baseline modulation, PPG amplitude modulation, and PPG frequency modulation. In one example, calculating SpO₂ microfluctuations comprises calculating a series of SpO₂ values over duration, where each SpO₂ values is calculated based on probe output data of less than or equal to one heartbeat of the subject, and identifying microfluctuations in SpO₂ over respiration period. SpO₂ microfluctuations may be used as an indication of one or more of the intravascular volume or cyclical recruitment of atelectasis. In another example respiration related SpO₂ microfluctuation may be used in parallel with respiration related PPG amplitude variation to get more reliable indicator of intravascular volume or hypovolemia status.

In examples, calculating the one or more physiological parameters includes calculating an indication of overall blood/intravascular volume. In examples, calculating the indication of overall blood volume comprises transforming the probe output to a frequency domain, determining an amplitude density at a respiratory frequency, and determining an amplitude density at a cardiac frequency. In one example, the overall blood volume is indicated to be low responsive to the amplitude density at the respiratory frequency being greater than a first threshold. In another example, calculating the indication of overall blood volume further comprises determining a ratio of the amplitude density at the respiratory frequency and the amplitude density at the cardiac frequency, wherein the overall blood volume is indicated to be low if the ratio is greater than a second threshold.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc., are used merely as labels and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims and may include other examples that occur to those of ordinary skill in the art. Such other examples are intended to be within the scope of the claims if they have structural elements that do not differ from the literal language of the claims or if they include equivalent structural elements with insubstantial differences from the literal languages of the claims.

1. A method of measuring arterial oxygen saturation (SpO₂) respiratory variability comprising:
generating a series of SpO₂ indications based on light transmission changes at a first wavelength and a second wavelength through blood originating from a carotid artery of a patient over a duration;
electronically measuring one or more respiration cycle microfluctuations in the series of SpO2 indications; and
displaying the one or more respiration cycle microfluctuations as an arterial blood oxygenation respiratory variability indicator.

2. The method of claim 1, wherein generating the series of SpO₂ indications comprises determining heart beat related relative light transmission changes at the first wavelength and the second wavelength.

3. The method of claim 1, wherein electronically measuring respiration cycle microfluctuations comprises:
identifying one or more values over a first threshold in the series of SpO₂ indications;
identifying one or more values under a second threshold in the series of SpO₂ indications; and
calculating differential values based on the one or more values over the first threshold and the one or more values under the second threshold of the SpO₂ indications to determine one or more respiration cycle microfluctuations in SpO₂, wherein each microfluctuation of the one or more respiration cycle microfluctuations is a differential value.

4. The method of claim 1, wherein the one or more respiration cycle microfluctuations are displayed numerically, visually, or audibly.

5. The method of claim 1, wherein each SpO₂ indication is measured over a second duration, wherein the second duration is less than or equal to a cardiac interbeat interval.

6. The method of claim 1, wherein the series of SpO₂ indications collectively represent a time period greater than or equal to a respiration period.

7. The method of claim 6, wherein the duration is greater than or equal to two respiration periods.

8. The method of claim 1, wherein prior to displaying the one or more respiration cycle microfluctuations as the arterial blood oxygenation respiratory variability indicator, the one or more respiration cycle microfluctuations in the series of SpO2 indications are converted to PaO₂ microfluctuations.

9. A system for an optical probe, comprising:
a plurality of light emitters at different wavelengths, and a light detector shaped to be attached on a nasal root of a patient and configured to measure light transmission through blood originating from an internal carotid artery of the patient and output a light transmission signal; and
a control and processing unit having instructions stored in memory or hardware configured to determine respiration cycle microfluctuation in arterial oxygen saturation based on extraction of plethysmograph waveforms from a plurality of the light transmission signals from the plurality of light emitters obtained over a duration, wherein the microfluctuation is a periodic SpO₂ change of less than 10% of SpO₂.

10. The system of claim 9, wherein a plurality of oxygen saturation values are measured during each respiration period, wherein a respiration period represents an amount of time elapsed for each inspiration and expiration cycle.

11. The system of claim 10, wherein each oxygen saturation value is measured for a second duration, wherein the second duration is shorter than or equal to a cardiac interbeat interval.

12. The system of claim 9, wherein the duration is longer than or equal to two respiration periods.

13. The system of claim 9, further comprising:
extracting a series of arterial oxygen saturation indications from the plethysmograph waveforms,
determining a plurality of variability values among the series of arterial oxygen saturation indications, wherein a variability value is a difference between a first arterial oxygen saturation indication and a second arterial oxygen saturation indication; and
determining respiration cycle microfluctuation as a representative value of the plurality of variability values measured over multiple respiration periods.

14. The system of claim 9, further comprising calculating a respiration rate of the patient by identifying a variation in probe output over the duration, wherein a frequency of the variation is the respiration rate.

15. The system of claim 9, wherein the control and processing unit is configured to use the microfluctuation in arterial oxygen saturation to determine a hypovolemia indicator.

16. The system of claim 15, wherein when an arterial oxygen saturation microfluctuation amplitude is greater than a threshold amount, hypovolemia is indicated.

17. The system of claim 15, wherein the control and processing unit is further configured to combine the microfluctuation in arterial oxygen saturation and PPG pulse amplitude variation to determine a hypovolemia indicator, wherein high pulse amplitude variation and high SpO₂ variation are indications of low intravascular volume status and low pulse amplitude variation and low SpO₂ variation are indications high intravascular volume status.

18. The system of claim 9, wherein the control and processing unit is configured to perform a first arterial oxygen saturation calculation for systole and a second arterial oxygen saturation calculation for diastole and determine the microfluctuation in arterial oxygen saturation based on a difference between the first and second arterial oxygen saturation calculations.

19. The system of claim 9, further comprising a display device, wherein the display device shows a trend in microfluctuations.

20. A system for an optical probe, comprising:
at least one light emitter and at least one light detector shaped to be attached on a nasal root of a patient and configured to measure light transmission through blood originating from an internal carotid artery of the patient and output a light transmission signal;
a control and processing unit including instructions to extract a respiration-related variation from at least one photoplethysmogram obtained from the at least one light transmission signal; and
a user interface unit configured to output the respiration related variation as an intravascular volume status indicator.
